(19) United States
(12) Patent Application Publication
(10) Pub. No.: US 2025/0250528 A1
(43) Pub. Date: Aug. 7, 2025

(54) INCUBATION DEVICE, INCUBATION SYSTEM AND SAMPLE ANALYZER

(71) Applicant: Shenzhen New Industries Biomedical Engineering Co., Ltd., Guangdong (CN)

(72) Inventors: Zexin LIAN, Guangdong (CN); Yingzu CHANG, Guangdong (CN); Liang ZHU, Guangdong (CN); Dingping BAN, Guangdong (CN); Hailin MU, Guangdong (CN)

(73) Assignee: Shenzhen New Industries Biomedical Engineering Co., Ltd., Guangdong (CN)

(21) Appl. No.: 19/022,191

(22) Filed: Jan. 15, 2025

(30) Foreign Application Priority Data
Feb. 2, 2024 (CN) 202410156368.0

(51) Int. Cl. C12M 1/00; C12M 1/34

(52) U.S. Cl. CPC C12M 41/14; C12M 23/02; C12M 23/38; C12M 41/46

(57) ABSTRACT

An incubation device, an incubation system and a sample analyzer are provided. The incubation device includes: a bearing portion, having a first bearing region and a second bearing region, wherein a first bearing hole is provided in the first bearing region, and a second bearing hole is provided in the second bearing region; and a cover plate structure, disposed on the second bearing region and having an opening position and a covering position. When the cover plate structure is at the covering position, the cover plate structure covers an upper side of the second bearing region, and when the cover plate structure is at the opening position, the cover plate structure avoids the second bearing region to expose the second bearing hole.

### CROSS-REFERENCE TO RELATED APPLICATION

The present disclosure claims priority to Chinese Patent Application No. 202410156368.0, filed to the China National Intellectual Property Administration on Feb. 2, 2024 and entitled “Incubation Device, Incubation System and Sample Analyzer”, the disclosure of which is hereby incorporated by reference in its entirety.

### TECHNICAL FIELD

The present disclosure relates to the field of medical instruments, and in particular to an incubation device, an incubation system and a sample analyzer.

### BACKGROUND

In the art, a sample analyzer is used for testing samples such as blood, and the sample analyzer usually includes a consumable box feeding system, a filling system, a reaction system, a measurement system and the like, wherein the reaction system is an indispensable component of the sample analyzer, and is configured to execute an incubation operation to provide a suitable reaction temperature for reactants in a reaction cup.

In the related art, for items that are prone to volatilization or evaporation in an incubation process, in order to avoid affecting other routine items, the incubation operation is generally performed separately outside the reaction system, for example, incubation is performed in a water bath box, after the incubation is completed, a worker places the reactants into the sample analyzer for testing, such that the operation is tedious, the workload of workers is relatively large, and errors are prone to occurring since more manual intervention is required.

### SUMMARY

The main objective of the present disclosure is to provide an incubation device, an incubation system and a sample analyzer, so as to solve the problem in the related art that items that are prone to volatilization or evaporation in an incubation process are separately incubated outside a reaction system, resulting in a tedious operation.

To achieve the above objective, according to one aspect of the present disclosure, provided is an incubation device, including: a bearing portion, having a first bearing region and a second bearing region, wherein a first bearing hole is provided in the first bearing region, and a second bearing hole is provided in the second bearing region; and a cover plate structure, disposed on the second bearing region and having an opening position and a covering position, wherein when the cover plate structure is at the covering position, the cover plate structure covers an upper side of the second bearing region, and when the cover plate structure is at the opening position, the cover plate structure avoids the second bearing region to expose the second bearing hole.

Further, a grabbing groove is provided in the second bearing region, and the second bearing hole is provided in a groove bottom wall of the grabbing groove.

Further, a structure of the second bearing hole is the same as a structure of the first bearing hole; and/or a plurality of first bearing holes are provided in the first bearing region in an array; and/or a plurality of second bearing holes are provided in the second bearing region in an array, a plurality of partition plates are disposed in the grabbing groove, the plurality of partition plates divide the grabbing groove into a plurality of mutually independent grabbing cavities, the plurality of grabbing cavities and the plurality of second bearing holes are disposed in one-to-one correspondence, a cross sectional area of the grabbing cavity is greater than a cross sectional area of a corresponding second bearing hole, and an upper surface of the partition plate is flush with an upper surface of the second bearing region.

Further, when a reaction cup is placed in the first bearing hole and the cover plate structure is at the covering position, an upper surface of the cover plate structure is flush with or lower than an upper surface of the reaction cup.

Further, the cover plate structure includes a rotating shaft penetrating through the bearing portion and a turning plate fixedly connected with the rotating shaft, and the rotating shaft is capable of driving the turning plate to turn over, so that the cover plate structure is switched between the covering position and the opening position.

Further, the incubation device further includes a position sensing structure, configured to detect a position of the cover plate structure.

Further, the position sensing structure includes a sensing groove and a sensing member disposed on the rotating shaft, and when the rotating shaft rotates, the rotating shaft drives the sensing member to switch between a state of extending into the sensing groove and a state of being outside the sensing groove.

Further, the incubation device further includes a driving portion, the driving portion includes a driving motor disposed below the bearing portion and a transmission structure disposed between the driving motor and the rotating shaft, and the transmission structure includes a first transmission wheel sleeved on an output shaft of the driving motor, a second transmission wheel sleeved on the rotating shaft, and a transmission belt sleeved outside the first transmission wheel and the second transmission wheel.

Further, the bearing portion includes a thermostatic member, a heating member and a thermal insulation layer, the first bearing hole and the second bearing hole are both provided on the thermostatic member, the heating member is disposed adjacent to the thermostatic member and is capable of heating the thermostatic member to 20° C.-50° C., and the thermal insulation layer covers a periphery of the thermostatic member and the heating member and a bottom of the thermostatic member and the heating member.

According to another aspect of the present disclosure, provided is an incubation system, including an incubation device and a reaction cup, wherein the incubation device is the incubation device mentioned above, and the reaction cup has a top opening and is capable of being placed in the first bearing hole or the second bearing hole.

According to another aspect of the present disclosure, provided is a sample analyzer, including the incubation device mentioned above or the incubation system mentioned above.

By applying the technical solutions of the present disclosure, the bearing portion is configured to bear the reaction cup and provide an appropriate incubation temperature for a sample in the reaction cup. The bearing portion includes the first bearing region, the first bearing hole is provided in the first bearing region, and the reaction cup can be placed in the first bearing hole to perform a routine item that is not prone to volatilization or evaporation; and the bearing portion further includes the second bearing region, the second bearing hole is provided in the second bearing region, the cover plate structure is disposed on the second bearing region and has the opening position and the covering position, when the cover plate structure is at the opening position, the cover plate structure avoids the second bearing region to expose the second bearing hole, at this time, the reaction cup can be placed in the second bearing hole, after the reaction cup is placed, the cover plate structure is switched from the opening position to the covering position, the cover plate structure covers the upper side of the second bearing region, so that the evaporation amount or volatilization amount of the sample in the reaction cup can be reduced, a concentration change of the sample is reduced, that is, the reaction cup can be placed in the second bearing hole to perform a special item that is prone to volatilization or evaporation. In the present application, the incubations of the routine item and the special item are integrated in one incubation device, the actions of placing the reaction cup in the first bearing hole or the second bearing hole and switching the cover plate structure between the opening position and the covering position can all be implemented by a machine without the need for manual operation of a worker, thereby reducing the labor intensity of the worker, and also avoiding misoperation caused by the introduction of the manual operation. Therefore, the technical solutions of the present application can effectively solve the problem in the related art that items that are prone to volatilization or evaporation in an incubation process are separately incubated outside the reaction system, resulting in a tedious operation.

### BRIEF DESCRIPTION OF THE DRAWINGS

The drawings, which constitute a part of the present application, are used to provide a further understanding of the present disclosure, and schematic embodiments of the present disclosure and descriptions thereof are used to explain the present disclosure, and do not constitute an improper limitation on the present disclosure. In the drawings:

FIG. 1 illustrates a schematic diagram of a three-dimensional structure of an embodiment of an incubation device according to the present disclosure, wherein a cover plate structure is at a covering position;

FIG. 2 illustrates an enlarged view of a position A of the incubation device in FIG. 1;

FIG. 3 illustrates a schematic top view of the incubation device in FIG. 1;

FIG. 4 illustrates a schematic sectional view of the incubation device in FIG. 1;

FIG. 5 illustrates an enlarged view of a position B of the incubation device in FIG. 4;

FIG. 6 illustrates a schematic sectional view of another position of the incubation device in FIG. 1;

FIG. 7 illustrates a schematic diagram of a three-dimensional structure of another angle of the incubation device in FIG. 1, wherein the cover plate structure is at an opening position;

FIG. 8 illustrates an enlarged view of a position C of the incubation device in FIG. 7;

FIG. 9 illustrates a schematic sectional view of an embodiment of an incubation system according to the present disclosure, wherein the cover plate structure is at the covering position; and

FIG. 10 illustrates an enlarged view of a position D of the incubation system in FIG. 9.

The above drawings include the following reference signs:
1. reaction cup; 1001. top opening; 1002. first cup body segment; 1003. second cup body segment; 1004. supporting edge;
10. bearing portion; 101. first bearing region; 102. second bearing region; 11. first bearing hole; 12. second bearing hole; 13. thermostatic member; 14. heating member; 15. thermal insulation layer; 16. grabbing groove; 161. grabbing cavity; 17. partition plate; 181. temperature sensor; 182, temperature control switch; 19. supporting member;
20. cover plate structure; 21. rotating shaft; 22. turning plate;
30. position sensing structure; 31. sensing groove; 32. sensing member; 33. sensing arm; 34. connecting arm;
40. driving portion; 41. driving motor; 42. transmission structure; 421. first transmission wheel; 422. second transmission wheel; 423. transmission belt; 43. motor frame.

### DETAILED DESCRIPTION OF EMBODIMENTS

A clear and complete description of technical solutions in the embodiments of the present disclosure will be given below, in combination with the drawings in the embodiments of the present disclosure. Apparently, the embodiments described below are merely a part, but not all, of the embodiments of the present disclosure. The following description of at least one exemplary embodiment is merely illustrative in nature and is in no way intended to limit the present disclosure and its application or usage. All of other embodiments, obtained by those ordinary skilled in the art based on the embodiments in the present disclosure without any creative effort, fall into the protection scope of the present disclosure.

It should be noted that, the terms used herein are for the purpose of describing particular embodiments only and are not intended to limit the exemplary embodiments in accordance with the present application. As used herein, singular forms are also intended to include plural forms, unless the context clearly dictates otherwise. In addition, it should also be understood that, when used in the present specification, the terms “containing” and/or “including” specify the presence of features, steps, operations, devices, components and/or combinations thereof.

Unless specifically stated otherwise, relative arrangement of components and steps, numerical expressions and numerical values set forth in these embodiments do not limit the scope of the present disclosure. In addition, it should be understood that, for ease of description, the sizes of portions shown in the drawings are not drawn according to actual proportional relationships. Techniques, methods and devices known to those ordinary skilled in the related art may not be discussed in detail, but where appropriate, the techniques, methods and devices should be considered as a part of the specification. In all examples shown and discussed herein, any specific value should be construed as exemplary only and not as a limitation. Thus, other examples of the exemplary embodiments may have different values. It should be noted that similar reference numerals and letters refer to similar items in the following figures, therefore once an item is defined in one figure, it is not necessary to be further discussed in the subsequent figures.

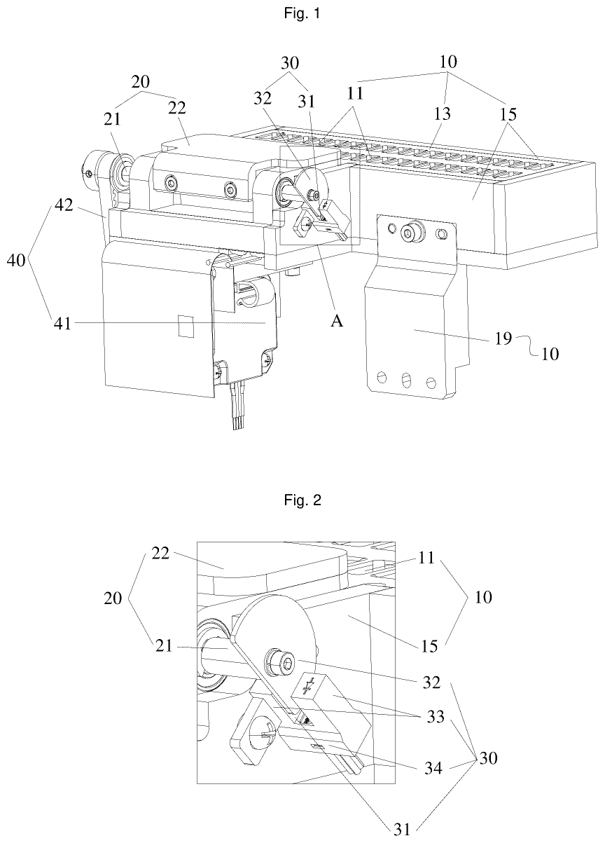

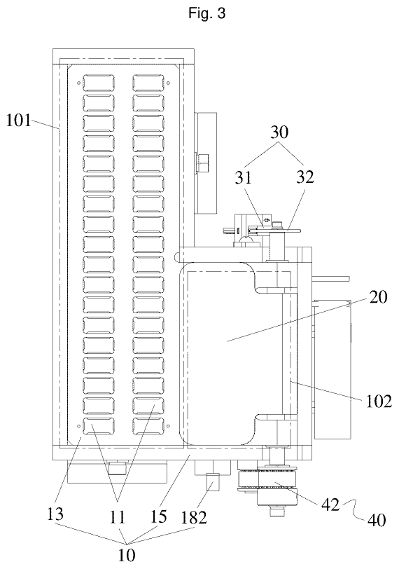

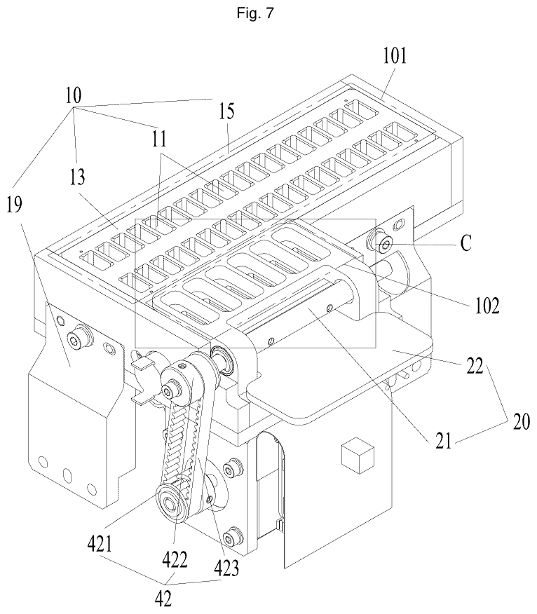

As shown in FIG. 1, FIG. 3 and FIG. 7, the present application provides an incubation device. An embodiment of the incubation device of the present application includes: a bearing portion 10 and a cover plate structure 20, wherein the bearing portion 10 has a first bearing region 101 and a second bearing region 102, a first bearing hole 11 is provided in the first bearing region 101, and a second bearing hole 12 is provided in the second bearing region 102; and the cover plate structure 20 is disposed on the second bearing region 102 and has an opening position and a covering position, when the cover plate structure 20 is at the covering position, the cover plate structure 20 covers an upper side of the second bearing region 102, and when the cover plate structure 20 is at the opening position, the cover plate structure 20 avoids the second bearing region 102 to expose the second bearing hole 12.

By applying the technical solutions of the present embodiment, the bearing portion 10 is configured to bear a reaction cup 1 and provide an appropriate incubation temperature for a sample in the reaction cup 1. The bearing portion 10 includes the first bearing region 101, the first bearing hole 11 is provided in the first bearing region 101, and the reaction cup 1 can be placed in the first bearing hole 11 to perform a routine item that is not prone to volatilization or evaporation; and the bearing portion 10 further includes the second bearing region 102, the second bearing hole 12 is provided in the second bearing region 102, the cover plate structure 20 is disposed on the second bearing region 102 and has the opening position and the covering position, when the cover plate structure 20 is at the opening position, the cover plate structure 20 avoids the second bearing region 102 to expose the second bearing hole 12, at this time, the reaction cup 1 can be placed in the second bearing hole 102, after the reaction cup 1 is placed, the cover plate structure 20 is switched from the opening position to the covering position, the cover plate structure 20 covers the upper side of the second bearing region 102, so that the evaporation amount or volatilization amount of the sample in the reaction cup 1 can be reduced, a concentration change of the sample is reduced, that is, the reaction cup 1 can be placed in the second bearing hole 12 to perform a special item that is prone to volatilization or evaporation. In the present embodiment, the incubations of the routine item and the special item are integrated in one incubation device, the actions of placing the reaction cup 1 in the first bearing hole 11 or the second bearing hole 12 and switching the cover plate structure 20 between the opening position and the covering position can all be implemented by a machine without the need for manual operation of a worker, thereby reducing the labor intensity of the worker, and also avoiding misoperation caused by the introduction of the manual operation. Therefore, the technical solutions of the present embodiment can effectively solve the problem in the related art that items that are prone to volatilization or evaporation in an incubation process are separately incubated outside a reaction system, resulting in a tedious operation.

When the present embodiment is described, the “routine item” is used to refer to an item that is not prone to volatilization or evaporation in the incubation process, and the “special item” is used to refer to an item that is prone to volatilization or evaporation in the incubation process.

For example, when a coagulation test is performed, the routine item needs to be incubated for 1 to 5 minutes in an environment of 37° C.; when items such as an APTT correction test is performed, long-time incubation is required, the worker generally completes 2 hours of incubation in a water bath box outside the incubation device, during this process, the sample is prone to evaporation, and thus the item is a special item. APTT is an abbreviation of activated partial thromboplastin time, which is a coagulation function test indicator capable of being used for reflecting an comprehensive activity of coagulation factors in an intrinsic coagulation pathway and especially in the first stage, and is widely used for screening and measuring defects of intrinsic pathway coagulation factors, such as factors XI, VIII and IX, and can also be used for primary screening diagnosis of bleeding diseases and laboratory monitoring of heparin anticoagulant therapy; and the correction test is also referred to as a mixing test or mixing study, in which related test items are retested after the plasma of a patient is mixed with mixed plasma of normal people in proportion. Performing the APTT correction test in the incubation device of the present embodiment, not only can reduce the evaporation amount in the incubation process, meanwhile since the incubation operations are performed in different bearing holes, when the item is incubated in the second bearing hole 12, the routine item can still be incubated in the first bearing hole 11, thereby not affecting the experimental process of the routine item.

Of course, the incubation device of the present embodiment is not only applicable to the APTT correction test that causes evaporation due to long-time incubation, but is also applicable to some other items in which samples or reagents have relatively high volatility themselves.

In addition, for tests such as the coagulation test, a doctor or an experimenter often needs to compare and analyze test results of the routine item and the special item to obtain a comprehensive analysis result, however, in the prior art, the routine item is performed in an automatic incubation device, and the special item is separately performed in the water bath box via the reaction cup with a cover, and due to the different incubation environments and transfer paths between the routine item and the special item, significant test errors are introduced, which proposes higher requirements for the experience of the doctor or the experimenter. In the present embodiment, the incubations of the routine item and the special item are integrated in one incubation device, so that other environmental features and reaction cup passing paths and the like are kept relatively consistent, which is beneficial to controlling variables, thereby facilitating the doctor or the experimenter to issue a trusted analysis result when comprehensively analyzing the routine item and the special item.

It should be noted that, at the covering position, the cover plate structure 20 can be in sealing fit with an upper surface of the second bearing region 102 or only presses against but does not seal the upper surface.

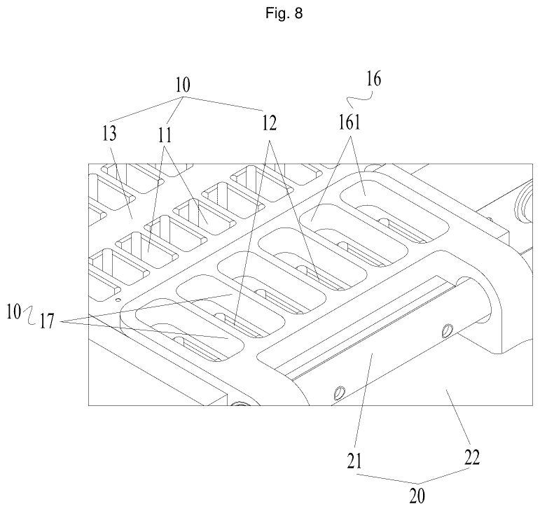

As shown in FIG. 7 and FIG. 8, a grabbing groove 16 is provided in the second bearing region 102, and the second bearing hole 12 is provided in a groove bottom wall of the grabbing groove 16. By providing the grabbing groove 16 and providing the second bearing hole 12 in the groove bottom wall of the grabbing groove 16, on one hand, an upper surface of the reaction cup 1 placed in the second bearing hole 12 can be lower than or flush with the upper surface of the second bearing region 102, so that the interior of the second bearing region 102 is relatively enclosed when the cover plate structure 20 covers the upper surface of the second bearing region 102, thereby avoiding or reducing the occurrence of evaporation or volatilization; and on the other hand, a grabbing mechanism for clamping the reaction cup 1 can extend into the grabbing groove 16 to place or take away the reaction cup 1.

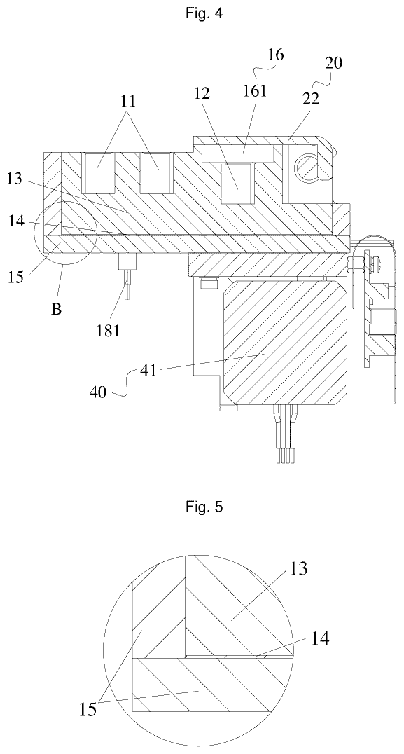

Specifically, as shown in FIG. 1, FIG. 3, FIG. 4, FIG. 5 and FIG. 7, the bearing portion 10 includes a thermostatic member 13, a heating member 14 and a thermal insulation layer 15, the first bearing hole 11 and the second bearing hole 12 are both provided on the thermostatic member 13, the heating member 14 is disposed adjacent to the thermostatic member 13 and is capable of heating the thermostatic member 13 to 20° C.-50° C., and the thermal insulation layer 15 covers a periphery of the thermostatic member 13 and the heating member 14 and a bottom of the thermostatic member 13 and the heating member 14, wherein the thermostatic member 13 is made from an aluminum alloy 6063-T5, the thermostatic member 13 includes a first thermostatic block and a second thermostatic block, which are integrally provided, a space occupied by the first thermostatic block forms the first bearing region 101, and a space occupied by the second thermostatic block forms the second bearing region 102; the heating member 14 is a PI heating film or a silicone rubber heating tape and is bonded below the thermostatic member 13; the thermal insulation layer 15 is thermal insulation cotton and covers the periphery of the thermostatic member 13 and the heating member 14 and the bottom of the heating member 14, so as to only expose an upper surface of the thermostatic member 13 to reduce the heat exchange between the bearing portion 10 and the outside, so that the thermostatic member 13 maintains a stable temperature.

It should be noted that, the incubation device in the present embodiment is not limited to be applied to the coagulation test in which incubation is performed at 37° C., and is also applicable to other items in which incubation is performed at relatively low temperatures (20° C. to 50° C.).

In the present embodiment, the first bearing region 101 and the second bearing region 102 share one thermostatic member 13 and one heating member 14, so that the cost can be saved on, the control of variables is facilitated, and it is helpful for the doctor to issue a trusted analysis conclusion.

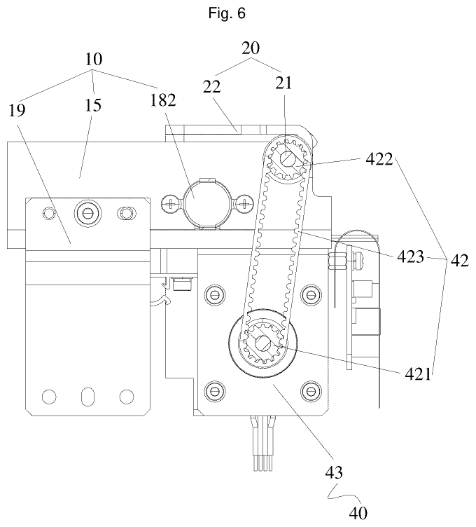

As shown in FIG. 3, FIG. 4, FIG. 6 and FIG. 7, the incubation device further includes a temperature sensor 181, a temperature control switch 182 and a supporting member 19, wherein the supporting member 19 is connected with the periphery of the bearing portion 10 and can be connected with a rack of a sample analyzer to fix the incubation device on the rack; the temperature sensor 181 is disposed in a central region of the thermostatic member 13 and can detect the temperature of the thermostatic member 13; and the temperature control switch 182 is electrically connected with both the temperature sensor 181 and the heating member 14, and when the temperature sensor 181 detects that the temperature of the thermostatic member 13 is greater than a preset value, the heating member 14 is controlled to stop heating.

As shown in FIG. 4, FIG. 7 and FIG. 8, a structure of the second bearing hole 12 is the same as a structure of the first bearing hole 11. In this way, the first bearing hole 11 and the second bearing hole 12 can match the reaction cups 1 of the same model number, therefore on one hand, the model numbers of the reaction cups 1 matching the incubation device are reduced, and on the other hand, the special item and the routine item can be incubated by using the reaction cups 1 of the same model number, thereby further reducing the introduction of errors during comprehensive comparison and analysis, so that the analysis result is more accurate.

As shown in FIG. 3, FIG. 7 and FIG. 8, a plurality of first bearing holes 11 are provided in the first bearing region 101 in an array, and a plurality of second bearing holes 12 are provided in the second bearing region 102 in an array. Since the plurality of first bearing holes 11 and the plurality of second bearing holes 12 are formed in the array, the path of the grabbing mechanism can be conveniently controlled. Specifically, 36 first bearing holes 11 are provided in the first bearing region 101 in a 2×18 manner, and 6 second bearing holes 12 are provided in the second bearing region 102 in a 1×6 manner. Of course, in specific implementation, the number of the first bearing holes and the array manner of the first bearing holes, and the number of the second bearing holes and the array manner of the second bearing holes are not limited to the above forms, but can be set by a designer as needed.

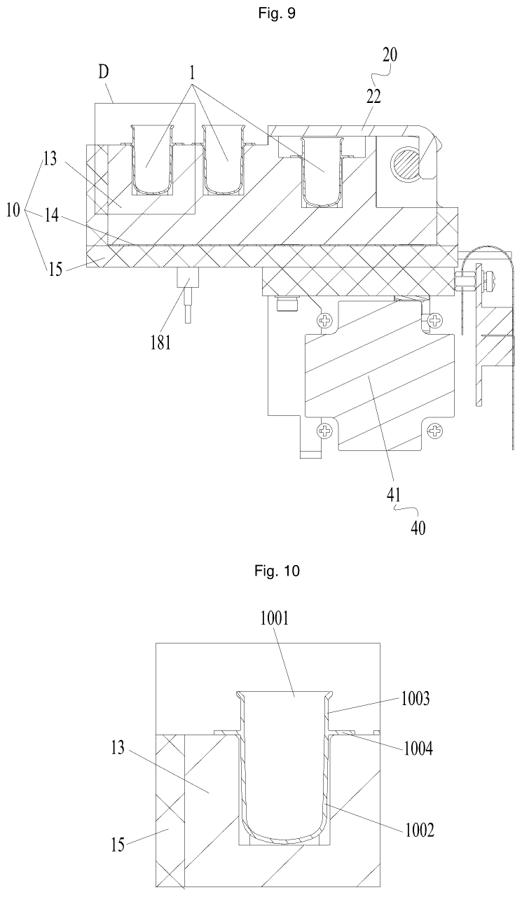

As shown in FIG. 4, FIG. 8 and FIG. 9, a plurality of second bearing holes 12 are provided in the second bearing region 102 in an array, a plurality of partition plates 17 are disposed in the grabbing groove 16, the plurality of partition plates 17 divide the grabbing groove 16 into a plurality of mutually independent grabbing cavities 161, the plurality of grabbing cavities 161 and the plurality of second bearing holes 12 are disposed in one-to-one correspondence, a cross sectional area of the grabbing cavity 161 is greater than a cross sectional area of a corresponding second bearing hole 12, and an upper surface of the partition plate 17 is flush with the upper surface of the second bearing region 102. By disposing the partition plates 17, when the cover plate structure 20 is at the covering position, the second bearing region 102 forms a plurality of mutually independent incubation spaces, and the sample in one reaction cup 1 can be incubated in each incubation space, so that the case where the accuracy of the test result is affected by the cross contamination of samples in the adjacent second bearing holes 12 during the incubation process can be reduced. Since the cross sectional area of the grabbing cavity 161 is greater than the cross sectional area of the corresponding second bearing hole 12, a grabbing space is formed between an upper end of the reaction cup 1 and a cavity wall of the grabbing cavity 161, so that the grabbing mechanism can conveniently extend into the grabbing space to place or take out the reaction cup 1.

As shown in FIG. 4 and FIG. 9, the second thermostatic block protrudes upwards from the first thermostatic block, a fence structure is formed between a circumferential side wall of the second thermostatic block and a circumferential side wall of the grabbing groove 16, and when the cover plate structure 20 is at the covering position, a lower surface of the turning plate 22 abuts against an upper surface of the fence structure and the upper surfaces of the partition plates 17, so that the second bearing region 102 forms a plurality of mutually independent incubation spaces.

As shown in FIG. 9, when the reaction cup 1 is placed in the first bearing hole 11 and the cover plate structure 20 is at the covering position, the upper surface of the cover plate structure 20 is flush with or lower than the upper surface of the reaction cup 1. In this way, when the incubations of the routine item and the special item are performed on the incubation device at the same time, the grabbing mechanism can normally run above the incubation device without the need for complicated movement such as avoidance.

As shown in FIG. 1 and FIG. 7, the cover plate structure 20 includes a rotating shaft 21 penetrating through the bearing portion 10 and a turning plate 22 fixedly connected with the rotating shaft 21, and the rotating shaft 21 is capable of driving the turning plate 22 to turn over, so that the cover plate structure 20 is switched between the covering position and the opening position. The structure for covering the upper side of the second bearing region 102 is configured as the turning plate 22, and the turning plate 22 is driven by the rotating shaft 21 to swing, thereby having the advantages of being simple in structure and reliable in control.

As shown in FIG. 1 to FIG. 3, the incubation device further includes a position sensing structure 30, configured to detect a position of the cover plate structure 20. The position sensing structure 30 can detect the position of the cover plate structure 20 to judge whether the cover plate structure 20 is at the covering position or the opening position.

Specifically, as shown in FIG. 1 and FIG. 2, the position sensing structure 30 includes a sensing groove 31 and a sensing member 32 disposed on the rotating shaft 21, and when the rotating shaft 21 rotates, the rotating shaft 21 drives the sensing member 32 to switch between a state of extending into the sensing groove 31 and a state of being outside the sensing groove 31. In the present embodiment, the position sensing structure 30 further includes two sensing arms 33 spaced apart from each other and connecting arms 34 connected with the end portions of the two sensing arms 33, the two sensing arms 33 and the connecting arms 34 jointly form the sensing groove 31, one of the two sensing arms 33 is capable of emitting an optical coupling signal, and the other one is capable of receiving the optical coupling signal. When the cover plate structure 20 is at the covering position, the sensing member 32 extends into the sensing groove 31 to block a transmission path of the optical coupling signal; and when the cover plate structure 20 is at the opening position, the sensing member 32 is located outside the sensing groove 31, so that the optical coupling signal can be smoothly transmitted. By judging whether the sensing arm 33 for receiving the optical coupling signal receives the optical coupling signal, the position of the cover plate structure 20 can be judged.

As shown in FIG. 3, FIG. 6 and FIG. 7, the incubation device further includes a driving portion 40, the driving portion 40 includes a driving motor 41 disposed below the bearing portion 10 and a transmission structure 42 disposed between the driving motor 41 and the rotating shaft 21, and the transmission structure 42 includes a first transmission wheel 421 sleeved on an output shaft of the driving motor 41, a second transmission wheel 422 sleeved on the rotating shaft 21, and a transmission belt 423 sleeved outside the first transmission wheel 421 and the second transmission wheel 422. Specifically, the driving motor 41 is a stepping motor, and the driving motor 41 drives the rotating shaft 21 and the turning plate 22 to rotate at a certain angle through the first transmission wheel 421, the second transmission wheel 422 and the transmission belt 423, so as to switch the position of the cover plate structure 20, thereby having the advantages of being simple in structure and easy to control. As shown in FIG. 5, the driving portion 40 further includes a motor frame 43, and the driving motor 41 is installed below the bearing portion 10 via the motor frame 43.

Of course, in embodiments not shown in the figures, the cover plate structure can also be configured as a structure for switching between the covering position and the opening position by translation, for example, the translation of a cover plate is implemented by a gear rack structure or a lead screw nut structure to close the second bearing region or avoid the second bearing region.

As shown in FIG. 9, the present application further provides an incubation system, an embodiment of the incubation system of the present application includes an incubation device and a reaction cup 1, the incubation device is the incubation device mentioned above, and the reaction cup 1 has a top opening 1001 and is capable of being placed in the first bearing hole 11 or in the second bearing hole 12. The above incubation device can effectively solve the problem in the related art that items that are prone to volatilization or evaporation in an incubation process are separately incubated outside a reaction system, resulting in a tedious operation, and the incubation system having the incubation device also has the above advantages.

Specifically, in the present embodiment, the incubation device includes a plurality of first bearing holes 11 and a plurality of second bearing holes 12, and there are a plurality of reaction cups 1, wherein at least part of the reaction cups 1 can be placed in the first bearing holes 11, and the other part of the reaction cups 1 can be placed in the second bearing holes 12. The first bearing holes 11 and the second bearing holes 12 have the same structures, therefore it is only necessary to provide reaction cups 1 of one model number.

As shown in FIG. 9 and FIG. 10, the reaction cup 1 includes a first cup body segment 1002, a second cup body segment 1003, and a supporting edge 1004, the first cup body segment 1002 is located below the second cup body segment 1003, the top opening 1001 is formed on the second cup body segment 1003, and the supporting edge 1004 is located at a junction of the first cup body segment 1002 and the second cup body segment 1003 and extends outwards. When the reaction cup 1 is placed in the first bearing hole 11, the upper surface of the first bearing region 101 is supported below the supporting edge 1004; and when the reaction cup 1 is placed in the second bearing hole 12, the groove bottom wall of the grabbing groove 16 is supported below the supporting edge 1004, and the second cup body segment 1003 is located in the grabbing cavity 161.

The present application further provides a sample analyzer, and an embodiment of the sample analyzer of the present application includes the above incubation device or the above incubation system. The above incubation device and the above incubation system can both effectively solve the problem in the related art that items that are prone to volatilization or evaporation in an incubation process are separately incubated outside a reaction system, resulting in a tedious operation, and the sample analyzer having the above incubation device or incubation system also has the above advantages.

In the description of the present disclosure, it should be understood that orientation or position relationships indicated by orientation words such as “front, back, upper, lower, left, right”, “transverse, longitudinal, vertical, horizontal” and “top, bottom” and the like are generally orientation or position relationships shown on the basis of the drawings, and are merely for the convenience of describing the present disclosure and simplifying the description, in the absence of opposite statements, these orientation words do not indicate or imply that the referred apparatuses or elements must have specific orientations or must be constructed and operated in specific orientations, and thus cannot be construed as limiting the protection scope of the present disclosure; and the orientation words “inside and outside” refer to the inside and outside of the contours of the components themselves.

For the convenience of description, spatially relative terms, such as “on”, “above”, “on the upper surface”, “over” and the like, may be used herein to describe spatial position relationships between one device or feature and other devices or features shown in the figures. It should be understood that, the spatially relative terms are intended to contain different orientations of a device in use or operation in addition to the orientations described in the figures. For example, if the device in the figures is turned over, the device, which is described as “above other devices or structures” or “over other devices or structures” would then be positioned as “below other devices or structures” or “beneath other devices or structures”. Thus, the exemplary term “above” may include both orientations of “above” and “below”. The device may also be otherwise positioned (rotated by 90 degrees or at other orientations), and the spatially relative descriptions used herein are interpreted accordingly.

In addition, it should be noted that the terms “first”, “second” and the like are used for defining components and parts, and are merely for the convenience of distinguishing the corresponding components and parts, and unless otherwise stated, the above words have no special meaning, and thus cannot be construed as limiting the protection scope of the present disclosure.

The above descriptions are only preferred embodiments of the present disclosure, and are not intended to limit the present disclosure. For those skilled in the art, the present disclosure may have various modifications and changes. Any modifications, equivalent replacements, improvements and the like, made within the spirit and principles of the present disclosure, shall be included within the protection scope of the present disclosure.

1. An incubation device, comprising:
a bearing portion, having a first bearing region and a second bearing region, wherein a first bearing hole is provided in the first bearing region, and a second bearing hole is provided in the second bearing region; and
a cover plate structure, disposed on the second bearing region and having an opening position and a covering position, wherein when the cover plate structure is at the covering position, the cover plate structure covers an upper side of the second bearing region, and when the cover plate structure is at the opening position, the cover plate structure avoids the second bearing region to expose the second bearing hole.

2. The incubation device according to claim 1, wherein a grabbing groove is provided in the second bearing region, and the second bearing hole is provided in a groove bottom wall of the grabbing groove.

3. The incubation device according to claim 2, wherein,
a structure of the second bearing hole is the same as a structure of the first bearing hole; and/or,
a plurality of first bearing holes are provided in the first bearing region in an array; and/or,
a plurality of second bearing holes are provided in the second bearing region in an array, a plurality of partition plates are disposed in the grabbing groove, the plurality of partition plates divide the grabbing groove into a plurality of mutually independent grabbing cavities, the plurality of grabbing cavities and the plurality of second bearing holes are disposed in one-to-one correspondence, a cross sectional area of the grabbing cavity is greater than a cross sectional area of a corresponding second bearing hole, and an upper surface of the partition plate is flush with an upper surface of the second bearing region.

4. The incubation device according to claim 1, wherein when a reaction cup is placed in the first bearing hole and the cover plate structure is at the covering position, an upper surface of the cover plate structure is flush with or lower than an upper surface of the reaction cup.

5. The incubation device according to claim 1, wherein the cover plate structure comprises a rotating shaft penetrating through the bearing portion and a turning plate fixedly connected with the rotating shaft, and the rotating shaft is capable of driving the turning plate to turn over, so that the cover plate structure is switched between the covering position and the opening position.

6. The incubation device according to claim 5, wherein the incubation device further comprises a position sensing structure, configured to detect a position of the cover plate structure.

7. The incubation device according to claim 6, wherein the position sensing structure comprises a sensing groove and a sensing member disposed on the rotating shaft, and when the rotating shaft rotates, the rotating shaft drives the sensing member to switch between a state of extending into the sensing groove and a state of being outside the sensing groove.

8. The incubation device according to claim 7, wherein the position sensing structure further comprises two sensing arms spaced apart from each other and connecting arms connected with end portions of the two sensing arms, the two sensing arms and the connecting arms jointly form the sensing groove, one of the two sensing arms is capable of emitting an optical coupling signal, and the other one is capable of receiving the optical coupling signal, and when the cover plate structure is at the covering position, the sensing member extends into the sensing groove to block a transmission path of the optical coupling signal; and when the cover plate structure is at the opening position, the sensing member is located outside the sensing groove, so that the optical coupling signal can be smoothly transmitted.

9. The incubation device according to claim 5, wherein the incubation device further comprises a driving portion, the driving portion comprises a driving motor disposed below the bearing portion and a transmission structure disposed between the driving motor and the rotating shaft, and the transmission structure comprises a first transmission wheel sleeved on an output shaft of the driving motor, a second transmission wheel sleeved on the rotating shaft, and a transmission belt sleeved outside the first transmission wheel and the second transmission wheel.

10. The incubation device according to claim 1, wherein the bearing portion comprises a thermostatic member, a heating member and a thermal insulation layer, the first bearing hole and the second bearing hole are both provided on the thermostatic member, the heating member is disposed adjacent to the thermostatic member and is capable of heating the thermostatic member to 20 C.-50° C., and the thermal insulation layer covers a periphery of the thermostatic member and the heating member and a bottom of the thermostatic member and the heating member.

11. The incubation device according to claim 10, wherein the thermostatic member comprises a first thermostatic block and a second thermostatic block, which are integrally provided, a space occupied by the first thermostatic block forms the first bearing region, and a space occupied by the second thermostatic block forms the second bearing region.

12. The incubation device according to claim 10, wherein the heating member is a PI heating film or a silicone rubber heating tape and is bonded below the thermostatic member.

13. The incubation device according to claim 10, further comprising a temperature sensor and a temperature control switch, wherein the temperature sensor is disposed in a central region of the thermostatic member and can detect a temperature of the thermostatic member, and the temperature control switch is electrically connected with both the temperature sensor and the heating member.

14. An incubation system, comprising an incubation device and a reaction cup, wherein the incubation device is the incubation device according to claim 1, and the reaction cup has a top opening and is capable of being placed in the first bearing hole or the second bearing hole.

15. The incubation system according to claim 14, wherein the reaction cup comprises a first cup body segment, a second cup body segment, and a supporting edge, the first cup body segment is located below the second cup body segment, the top opening is formed on the second cup body segment, and the supporting edge is located at a junction of the first cup body segment and the second cup body segment and extends outwards.

16. The incubation system according to claim 14, wherein a grabbing groove is provided in the second bearing region, and the second bearing hole is provided in a groove bottom wall of the grabbing groove.

17. The incubation system according to claim 16, wherein,
a structure of the second bearing hole is the same as a structure of the first bearing hole; and/or,
a plurality of first bearing holes are provided in the first bearing region in an array; and/or,
a plurality of second bearing holes are provided in the second bearing region in an array, a plurality of partition plates are disposed in the grabbing groove, the plurality of partition plates divide the grabbing groove into a plurality of mutually independent grabbing cavities, the plurality of grabbing cavities and the plurality of second bearing holes are disposed in one-to-one correspondence, a cross sectional area of the grabbing cavity is greater than a cross sectional area of a corresponding second bearing hole, and an upper surface of the partition plate is flush with an upper surface of the second bearing region.

18. The incubation system according to claim 14, wherein when a reaction cup is placed in the first bearing hole and the cover plate structure is at the covering position, an upper surface of the cover plate structure is flush with or lower than an upper surface of the reaction cup.

19. A sample analyzer, comprising the incubation device according to claim 1.

20. A sample analyzer, comprising the incubation system according to claim 14.
